(19) United States
(12) Patent Application Publication
(10) Pub. No.: US 2019/0321824 A1
(43) Pub. Date: Oct. 24, 2019

(54) MAGNETIC BIOSENSOR DESIGN FOR MANUFACTURING

(71) Applicants: Igor IZYUMIN, Oakland, CA (US); Octavian FLORESCU, Carlsbad, CA (US)

(72) Inventors: Igor IZYUMIN, Oakland, CA (US); Octavian FLORESCU, Carlsbad, CA (US)

(21) Appl. No.: 16/460,480

(22) Filed: Jul. 2, 2019

Related U.S. Application Data
(63) Continuation of application No. PCT/US2018/012357, filed on Jan. 4, 2018.
(60) Provisional application No. 62/443,535, filed on Jan. 6, 2017.

(51) Int. Cl. B01L 3/00; G01N 33/53; G01N 33/543; G01N 21/77; H05K 1/18; H05K 1/02

(52) U.S. Cl. CPC B01L 3/502715; H05K 2201/10121; G01N 33/54333; B01L 3/502761; B01L 3/502707; G01N 21/77; H05K 1/185; H05K 1/0272; B01L 2200/0668; B01L 2300/0636; B01L 2300/0681; B01L 2400/0406; B01L 2400/043; B01L 2200/143; H05K 2201/10151; G01N 33/5302

(57) ABSTRACT

A stand-alone assay system for POC IVD that is fast, easy to use, inexpensive and delivers laboratory quality results for one or more target analytes from a small volume of unprocessed aqueous sample is disclosed. The stand-alone assay system is a disposable digital device. The stand-alone assay system integrates a sampling system that relies on users swiping a lanced finger containing an unprocessed aqueous sample such as whole blood across a sample inlet to transfer or deposit the unprocessed aqueous sample on to a membrane filter or into a capillary. The system can have a planarized testing surface on which retained magnetic particles are sensed.

### CROSS-REFERENCE TO RELATED APPLICATION

The present application is a continuation of International Application No. PCT/US2018/012357, filed Jan. 4, 2018, which claims priority to U.S. Provisional Application No. 62/443,535, filed Jan. 6, 2017, both of which are incorporated by reference herein in their entireties.

### BACKGROUND

### 1. Technical Field

An assay system and method for use in the field of chemical testing is disclosed. More particularly, the assay system can be used for analyzing particles in fluid samples on a planarized analysis surface.

### 2. Summary of the Related Art

Point-of-Care (POC) diagnostic medical devices facilitate early stage detection of diseases, enable more individually tailored therapies, and allow doctors to follow up with patients more easily to see if prescribed treatments are working. To ensure widespread adoption, these tools must be accurate, easy to use by untrained individuals, and inexpensive to produce and distribute. Immuno-Assay (IA) applications are particularly well-suited for the POC since a wide range of conditions, from cardiovascular disease to cancer to communicable infections, can be identified from soluble protein bio-markers. The detection and quantitation of these bio-markers from raw samples such as whole blood often involves labeling the target protein using fluorescent or phosphorescent molecules, enzymes, quantum dots, metal particles or magnetic particles. For high sensitivity applications, the labels specifically bound to the target analytes must be distinguished from the unbound ones that contribute to background noise. By combining both label separation and detection in a low cost, easy to use format, the Immuno-Chromatographic Test (ICT) achieves stand-alone operation, i.e. the ability to perform an assay without necessitating an electronic reader or an external sample preparation system. Stand-alone operation is an often overlooked attribute, but one that is key to the popularity of ICTs, achieved despite other drawbacks such as low biochemical sensitivity, user interpretation, inaccurate quantitation, timing requirements, and awkward multiplexing.

The use of magnetic particle labeling is ideal for POC applications; magnetic particles can be individually detected, so sub-picomolar sensitivities can be achieved without signal amplification steps that can take up to an hour as in case of enzymatic labeling. Also, by micro-arraying the sensing onto which the particles bind, multiplexed operation can be achieved at low cost. The use of magnetic particles can reduce incubation times, since they can bind to the target analytes with solution-phase kinetics due to their high surface area to volume ratio. Furthermore, the ability to pull the magnetic particles out of solution magnetically and gravitationally overcomes the slow diffusion processes that plague most high sensitivity protocols. The signals from magnetic particles can be stable over time, insensitive to changes in temperature or chemistries and detected in opaque or translucent solutions like whole blood or plasma. The biological magnetic background signal can be low, so high assay sensitivity can be achieved with minimal sample preparation. Importantly, the use of magnetic particles as assay labels can permit stand-alone device operation, since these particles can be both manipulated and detected electromagnetically.

Magnetic particles are nano-meter or micro-meter sized particles that display magnetic, diamagnetic, ferromagnetic, ferrimagnetic, paramagnetic, super-paramagnetic or antiferromagnetic behavior. Magnetic particles can refer to individual particles or larger aggregates of particles such as magnetic beads.

Magnetic particle sensors are sensors embedded in an integrated circuit that can detect magnetic particles. Examples include optical sensors, magnetic sensors, capacitive sensors, inductive sensors, pressure sensors, or microbalance (mass) sensors.

One means for integration into a stand-alone assay system is to use magnetic particles that bind to the target analytes in solution before sedimenting via gravity or magnetic force to sensing areas where the specifically bound particles can be detected. A bio-functionalized IC can be used to detect the specifically bound particles. However, most IC-based immuno-assay implementations reported to date cannot operate stand-alone since they require either off-chip components for particle detection, or micro-fluidic actuation for particle manipulation and sample preparation. Other implementations simply cannot reach the cost structures necessary to compete in the current marketplace.

For POC application, it is desirable that the sample preparation be rapid since the assay is limited to 10-15 minutes. In addition, to obviate the need for refrigeration equipment and to facilitate storage and distribution, a dry sample preparation system is desired. It is also desirable to have a sample preparation system that receives small unprocessed samples from patients. The average hanging drop of blood from a finger stick yields approximately 150 of fluid. For more fluid, a complicated venu-puncture can be necessary. Moreover, the sample preparation system must be low-cost since biological contamination concerns dictate that all material in contact with biological samples be discarded. It is also desirable that the sample preparation system be amenable to multiplexed operation.

A porous material like a membrane filter can obviate the need for centrifugation or complicated micro-fluidic sample preparation. Since the membrane filters are compact and inexpensive, system cost is reduced, enabling stand-alone POC operation. Furthermore, the membranes can separate the plasma from the whole blood cells without additional support in under 30 seconds. Incubation of the filtrate with functionalized magnetic particles can achieve solution phase kinetics for rapid operation with sub pico-molar sensitivities. The use of an IC to perform the detection of the magnetic particles enables low cost, stand-alone operation. Therefore, the combination of a filter, capillary, magnetic particles and an IC can result in a stand-alone, accurate, multiplexed platform with the form factor of a thumb-drive.

The assay system can be a battery operated stand-alone device and can contain a digital display to display the results. Results can also be wirelessly transmitted to a receiver device such as a mobile phone, a personal computer, or a specialized reader unit.

### BRIEF DESCRIPTION OF THE DRAWINGS

FIG. 1 is a cross-sectional representation of the biosensor assay system.

FIG. 2 shows the top view of the IC 1 with magnetic particles on the surface 6. The adjacent sensors in the center are clumped sensors since they all detect magnetic particle simultaneously.

FIG. 3 is a cross-sectional representation of one of the steps of the planarization process, namely when the PCB 2 and the IC 1 are mounted or fitted to the planarization surface 51.

FIG. 4 is a cross-sectional representation of the interface between the IC 1, the PCB 2 and the fluid unit 12.

### DETAILED DESCRIPTION

The design for manufacturing of a magnetic biosensor assay system is disclosed. The assay system can be used for immuno-assays. The assay system can be used for nucleic acid, small molecule and inorganic molecule detection, or combinations thereof.

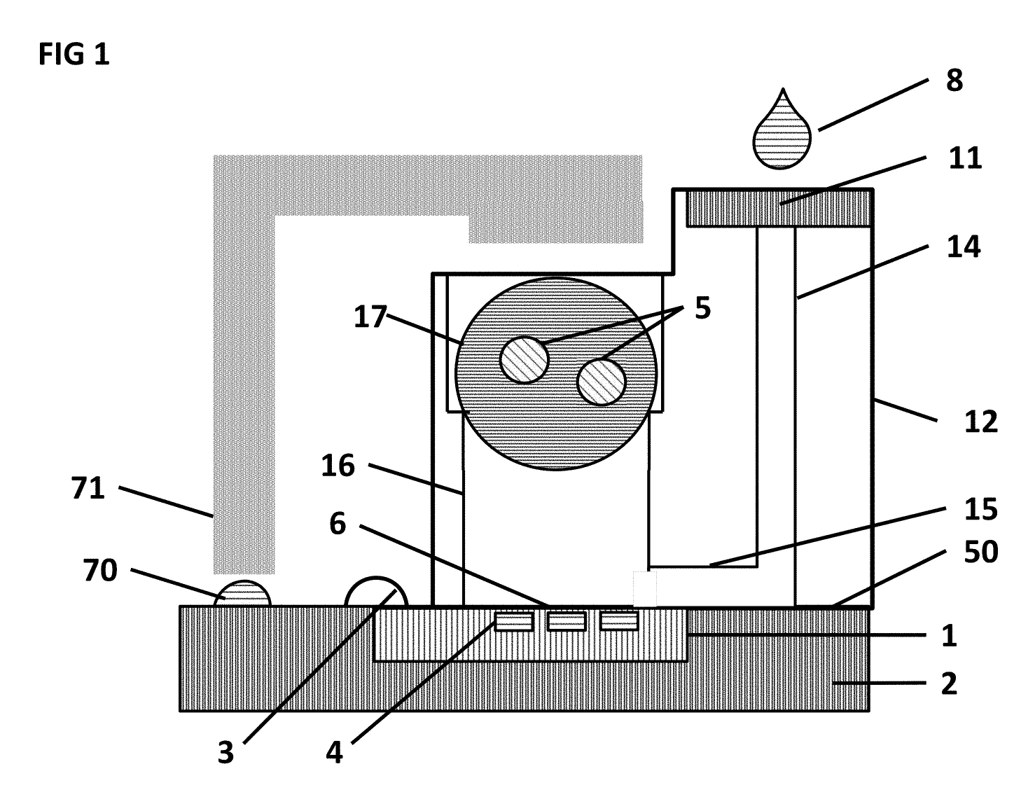

FIG. 1 presents a cross-sectional side view of the magnetic biosensor assay system, which can consist of an Integrated Circuit (IC) 1 mounted onto a Printed Circuit Board (PCB) 2. Wirebonds 3 can electrically connect the pads on the IC 1 to the traces on the PCB 2. FIG. 1 omits the battery, display and plastic outer housing.

The IC 1 can contain magnetic particle sensors 4 for detecting magnetic particles 5 specifically bound to the functionalized surface 6 of the IC 1. The system, for example in a biosensing format, can detect magnetic particles 5 that bind strongly and/or specifically to the surface 6 of the integrated circuit 1 as a result of one or more chemical or biochemical reactions involving one or more target analyte 7 in an aqueous sample 8. The magnetic particles 5 can be coated or functionalized with reagents that react with the target analyte 7. The surface 6 of the IC 1 can be coated or functionalized with reagents that react with the target analyte 7. The number of magnetic particles 5 specifically bound to the surface 6 of the IC 1 is representative of the concentration of the target analyte 7 in the aqueous sample 8 presented. The assay format can be competitive or capture.

The magnetic particles sensors 4 can be optical, magnetic acoustic or other. An array of magnetic particle sensors 4 can produce a raw image of magnetic particles 5 on the surface 6 of the IC 1. The raw image can be process, filtered and manipulated to yield the appropriate magnetic particle count and ultimately concentration.

For optical sensors 4, a light source 18 can provide illumination to the surface 6 of the IC 1 vertically through the sedimentation capillary 16. The optical sensors can detect the amount of incident light. Magnetic particles 5 can cast a shadow vertically downward and reduce the amount of light incident on a sensor 4. The sensors 4 can measure the amount of incident light to determine whether a magnetic particle 5 is present on the surface above it.

The magnetic particles 5 can be attracted to the sensors 4 positioned under the biologically coated, functionalized surface 6 of the integrated circuit 1 by magnetic concentration forces that can be generated by current passing through concentration conductors 9 embedded in the integrated circuit 1. The magnetic particles 5 that react with the target analyte 7 can bind specifically to the surface 6 of the integrated circuit 1 exposed to the aqueous sample 8. The magnetic particles 5 that do not react with the target analyte 7 can bind non-specifically to the surface 6 of the integrated circuit 1 and can be removed from above the sensors 4.

The non-specifically bound magnetic particles 5 can be removed from the sensors 4 positioned under the biologically coated, functionalized surface 6 of the integrated circuit 1 by magnetic separation forces that can be generated by current passing through separation conductors 10 embedded in the integrated circuit 1. One or more magnetic separation conductors 10 embedded in the integrated circuit can produce one or more magnetic separation forces. The magnetic separation forces can remove the non-specifically bound magnetic particles 5 from the sensors 4, such that only the specifically bound magnetic particles 5 remain.

A fluidic unit 11 consists of a membrane filter 12 and one or more capillaries configured to deliver magnetic particles 5 to the exposed surface 6 of the IC 1. The large particulate matter in the sample, such as whole blood cells, can be trapped on top or in the membrane, while the aqueous sample 8 containing the target analytes 7 can traverse the membrane filter 12 and can flow into a capillary where dry magnetic particles 5 can be re-hydrated. The re-hydrated magnetic particles 5 can bind to the target analytes 7 in the filtrate 13. The magnetic particles 5 that bound or reacted with the target analytes 7 can sediment and bind specifically to the surface 6 of the IC 1.

The fluidic unit 11 can consist of multiple capillaries, for example a delivery capillary 14, a surface capillary 15 and a sedimentation capillary 16. A delivery capillary 14 can be placed directly below the membrane filter 13 can and be fluidically connected to a surface capillary 15. The delivery capillary 14 can deliver the filtrate 13 into the surface capillary 15. The surface capillary 15 can be placed directly above the surface 6 of the IC 1 and can be fluidically connected to one or more sedimentation capillary 16. The surface capillary 15 can deliver the filtrate 13 into one or more sedimentation capillary 16.

The one or more sedimentation capillaries 16 can be placed vertically directly above the sensors 4. Dried magnetic particles 5 can be placed at the top of the sedimentation capillary 16. The magnetic particles can be dried into a dry sphere 17 through a lyophilization process. From the top of the sedimentary capillary 16, the dried magnetic particles 5 can be re-hydrated and they can sediment to the sensors once the filtrate 13 reaches them. The incubation time of the assay can be determined by the height of the sedimentation capillary 16.

The number of specifically bound magnetic particles 5 detected by the sensors is an indication of the concentration of the target analytes 7 in the filtrate 13. The assay system may be configured to take whole or previously filtered blood, urine, tear, sputum, fecal, oral, nasal samples or other biological or non-biological aqueous samples. The system can be assembled without a membrane filter 12, and the aqueous sample 8 can be introduced directly into the delivery capillary 14.

Chemicals, such as, but not limited to: aptamers, oligonucloetides, proteins, agents to prevent clotting, target analytes for internal calibration curves, bindive catalytic agents, magnetic particles, or combinations thereof may be dried in the membrane filter assembly along the shaft of the capillary or on the surface of the IC and can be re-solubilized by the blood plasma but remain bound to the surface upon which they were dried.

Several processes may interfere with the reliable detection of magnetic particles 5 on the surface 6 of the IC 1. Such processes can include light scattering by suspended magnetic particles 5, debris, gas bubbles, sample matrix effects, excipients that have not completely dissolved, and light scattering due to variations in the refraction index, or reflections from sidewalls of the sedimentation capillary. These effects can cause the light incident multiple sensors 4 to be substantially non-uniform in intensity and angle of incidence. The detection algorithm for detecting magnetic particles 5 can account for these effects, since the variation due to illumination non-uniformity may be significantly larger than the variation due to reduction of incident light by the shadow cast by the magnetic particle 5. Furthermore, the illumination non-uniformity may vary over time due to diffusion, convection, dissolution, and brownian motion of the filtrate 13 or of magnetic particles 5 in the filtrate 13. Therefore, a simple calibration of the illumination before starting the assay is insufficient to ensure reliable detection of magnetic particles.

The present disclosure addresses these problems by dynamically estimating the background illumination for detection, and dynamically adjusting the effective sensor gain to compensate for non-uniform background illumination. The background illumination can be reliably estimated by observing that shadows due to magnetic particles 5 on the surface 6 of the IC 1 have a much higher spatial frequency than shadows or illumination variations due to particles, contaminants, and reflections significantly above the surface 6 (relative to the size of the sensor opening), because the latter are subject to diffraction and other scattering processes. Thus, the background illumination may be reliably estimated by an appropriate estimator. The specific design of such an estimator may vary significantly due to system constraints and computational resource availability. A particularly simple estimator that results in good performance can entail signal filtering the raw image using a rectangular moving average filter. The parameters of the signal filter can be appropriately chosen to ensure sufficient attenuation of signals due to magnetic particles 5 on the surface 6, while still removing most of the background illumination. For example, a 3×9 moving average window is sufficient in a particular embodiment of the disclosure. Other, more complex or nonlinear signal filters may be used to achieve the same effect. For example, the moving average filter can be replaced with a median filter, or a more complex convolution kernel can be used instead of a rectangular window, such as a Gaussian filter. These approaches can potentially obtain somewhat better performance at the cost of more computational resources.

Sensor gain for each sensor 4 pixel can be calculated from the estimated background illumination. The low-frequency background illumination components can be removed from the raw image by scaling the detected brightness for each pixel of the sensor 4 by the reciprocal of the estimated background illumination at that pixel, resulting in a processed image with substantially uniform background illumination. It is important to note that the processed image may still contain variations due to components with a high spatial frequency. The main such component is fixed-pattern noise of the sensor 4 which may result due to e.g. variations in photodiode capacitance or other hardware artifacts. However, the components with a high spatial frequency are independent of the optical characteristics of the sedimentation capillary 16, and can therefore be effectively removed with a factory calibration, or a calibration at the beginning of the assay procedure.

High spatial frequency calibration may be performed by calculating the background-subtracted processed image as described in the preceding text, and calculating the effective pixel gain as the reciprocal of the processed image value. If this is done when no (or few) particles 5 are present on the sensor 4, the resulting gain represents the light sensitivity of the sensor 4 pixel itself, with the background illumination variations removed. Since this gain is a function primarily of the physical characteristics of the individual sensor 4 pixel, they are largely time-invariant, and can thus be calculated prior to the assay being initiated. This table can therefore be generated at the factory, and stored in non-volatile memory for later use. In an alternate embodiment, this calibration may be performed upon power-up, before particles 5 have reached the sensor 4 surface.

It is important to verify that the assay process is proceeding as intended, and the high-resolution detection permitted by individual magnetic particle sensors 4 allows many such checks to be implemented. The first error source is variations in the total number of magnetic particles 5 in the system as measured by the density of the magnetic particles 5 on the sensor 4 prior to magnetic separation. Such variations could arise due to manufacturing variations and other factors that are difficult to control. While ratiometric detection (i.e. detecting the magnetic particles 5 before and after magnetic separation and calculation a binding ratio) is nominally insensitive to the absolute magnetic particle 5 count, there are several second-order effects that must be considered. First, an insufficient number of magnetic particles 5 on the sensor 4 prior to magnetic separation could result in an abnormally high coefficient of variation for the assay, potentially bringing it out of specification and yielding an incorrect result. If an abnormally high number of magnetic particles 5 is present, magnetic separation effectiveness can be compromised. Furthermore, depending on the kinetics of the analyte 7 and the properties of the capture antibody, variations in the magnetic particle 5 count can shift the calibration curve of the assay due to the magnetic particles 5 partially depleting the sample 8, resulting in fewer target analyte 7 molecules binding to capture sites on the magnetic particles 5 and thus decreasing the binding ratio.

The present disclosure alleviates these problems by incorporating quality control metrics into the assay. If the magnetic particle 5 surface density on the surface of the IC 1 is abnormally low or high, the assay will be aborted and an error will be indicated to the user. If the magnetic particle 5 surface density is within an acceptable range, an appropriate calibration curve can be selected for transforming the binding ratio to a final analyte concentration or assay indication. Alternatively, the error may be corrected by an appropriate auxiliary calibration adjustment, or by interpolation between two or more calibration curves.

Another source of error is variations in the magnetic particle 5 surface density distribution across the surface of the sensor 4. Such variations may occur due to bubbles trapped in the sedimentation capillary 16, which tend to push sedimenting magnetic particles 5 to the sides of the sedimentation capillary 16, resulting in variations in the magnetic particle 5 surface density.

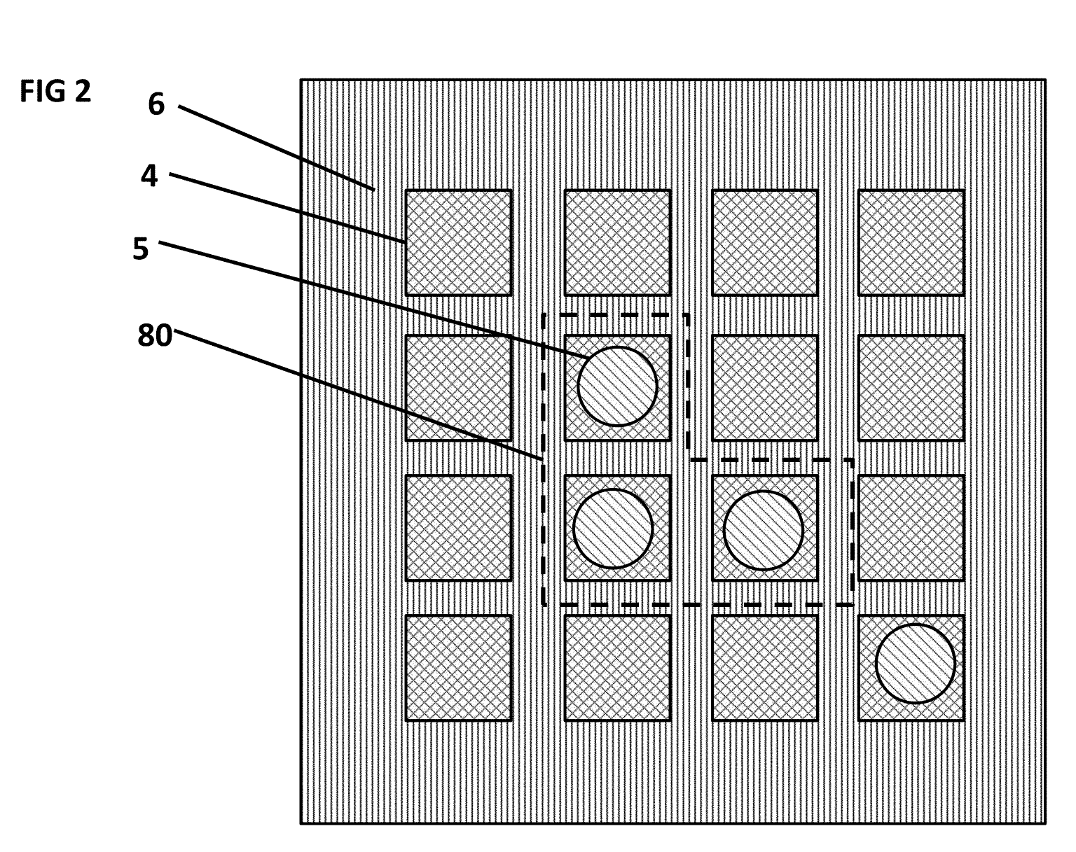

Another source of error is formation of clumps of magnetic particles 5, which may occur during the drying process. Clumps of magnetic particles cannot be effectively separated magnetically and do not behave the same way as monodisperse magnetic particles, so they must be excluded from analysis. This may be done by excluding from any analysis adjacent sensors that detect a magnetic particle, defined as clumped sensors 80. FIG. 2 shows a top view of the IC 1 with 16 sensors. The outline of the clumped sensors 80 shows adjacent sensors detecting magnetic particles. The data from clumped sensors 80 can be excluded from the before magnetic separation images (raw or processed images) or the after magnetic separation images (raw or processed images) to avoid corrupting the analytical measurement. The clumped sensors can be defined as 2, 3, 4, 5 or more adjacent sensors that detect a magnetic particle on their surface simultaneously. The selection of the number of adjacent sensors to exclude may be performed by first considering the nominal size of the clump in the X and Y directions direction, and then excluding adjacent sensors within a certain sensor radius. Because the sensor spacing may vary substantially in in the X and Y directions, it may be necessary to only consider horizontal or vertical clumps, or to use a different radius in the X and Y directions. The nominal size of the clumps can be determined at run time to incorporate matrix effects. This method enables the ability to discretely eliminate magnetic particle clumps from the measurement.

Another quality control check that is useful in detecting abnormal magnetic particle 5 distribution is measuring the spatial density of magnetic in a given sensor 4 area, and computing the moving window of this density. Sensor areas with a substantially higher or lower magnetic particle density may indicate an artifact. If the variation is excessive, the assay may be aborted and an error indicated, or the sensor area may be excluded.

The accurate computation of the binding ratio requires measuring the ratio of the number of magnetic particles 5 that stay bound after magnetic separation to the number of magnetic particles 5 that were present before the magnetic separation. A sedimentation error can be introduced if magnetic particles are sedimenting while the wash is occurring. This sedimentation error may be rejected by using the image from before the magnetic separation as an inclusion mask for the image from after magnetic separation. Only sensors that detected magnetic particles on the surface before magnetic separation are included in the detection of particles after magnetic separation. The inclusion mask excluding any newly landed magnetic particles that landed onto other sensor pixels during magnetic separation which can take up to several minutes.

To ensure the entire sedimentation capillary 16 aperture is always positioned over the sensor area, it may be necessary to make the total sensor 4 area larger than the cross section area of the sedimentation capillary 16 plus any alignment tolerances. This implies that part of the sensor 4 will be outside the aperture of the vertical sedimentation capillary 16, and will receive less illumination. This region may be excluded from analysis, because the background illumination estimator process may increase the sensor gain in the darker region substantially, causing sensor noise to be amplified and potentially resulting in false magnetic particle detection. In an embodiment, the dark region can be computed by the following method: the median sensor gain of an unobstructed sensor is estimated by finding the median of all the sensor gain values. If a large number of sensors are expected to be occluded, another percentile can be used in place of the median (e.g. 80th percentile). The minimum and maximum sensor gains can then be calculated by multiplying the median sensor gain by an upper and lower adjustment factors, which are set to account for sensor gain distribution. In an embodiment, the lower and upper factors may be set to 0.6 and 1.4, respectively. The sensors whose sensor gain falls outside this range may form an exclusion zone, which is stored as a binary exclusion image where the “1” value represents an excluded sensor. This exclusion image may then morphologically dilated several times to expand the exclusion zone. In an embodiment, the number of dilations may be set to 1, 2, 3, 4, 5 or more. The dilation operation may expand the exclusion zone so that sensors close to an edge or artifact are excluded, since those sensors are disproportionately affected by edge effects and illumination changes. This operation may also fill small “holes” in the exclusion zone that can occur if e.g. a defective sensor is located in an occluded area. Optionally, single isolated exclusion sensors having no neighbors can be removed prior to dilation, and the dilated bitmap can be logically ORed with the original exclusion image. This avoids expanding the exclusion area around single defective sensors.

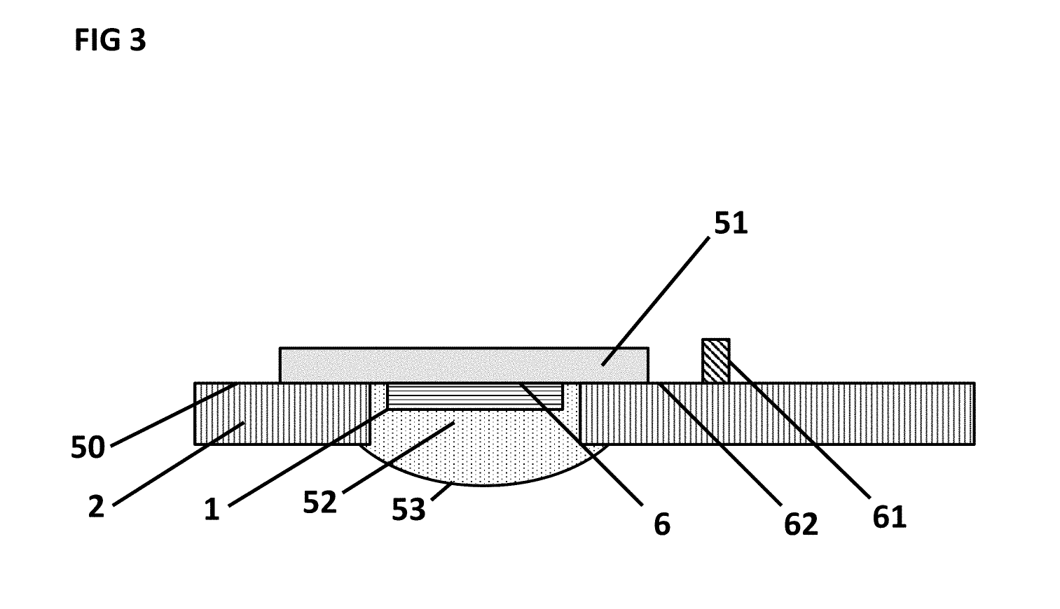

In order to minimize sensor cost, it may be necessary to minimize the area required by the integrated circuit 1. FIG. 3 shows the cross section of a planarization process that can place the surface 6 of the IC 1 substantially into the plane with the surface 50 of the PCB 2, thus permitting the PCB 2 and IC 1 to seal with the fluidic unit 12. The planarization process may involve placing the IC 1 in a cutout 52 or pocket made into the PCB 2. The IC 1 may be placed with its surface 6 against a planarization surface 51. The planarization surface 51 can extend beyond the outline of the cutout 52, thus putting the surface 6 of the IC 1 and the surface 50 of the PCB 2 substantially into the same plane. The IC 1 may be physically attached to the planarization surface 51 via a temporary adhesive, held by surface forces or friction, and/or held by vacuum or mechanical means. The cutout 52 can then be back-filled with a liquid polymer resin 53 (such as epoxy resin, or another suitable material), which is then allowed to solidify, or other filler material. Pressure or vacuum may be used to assure complete and void-free filling of the cutout 52. The planarization surface 51 can then be removed, thus yielding a planarized circuit assembly where the surface 6 of the IC 1 is coplanar with the surface 50 of the PCB 2 and with the resin 53.

Ideally, the surface planarity of the IC surface 6 and the PCB surface 50 and the resin 53 is less than 20 um, 15 um, 10 um, Sum, 3 um, 2 um, 1 um. To achieve such tight tolerance, the distance from the edge of the IC 1 to the side of the cutout 52 in the PCB 2 may be less than 1 mm, 0.5 mm, 0.25 mm, 0.2 mm, 0.1 mm, 50 um, 25 um, 10 um, 5 um, 1 um. This is especially important if the planarization surface 51 is not rigid.

In one embodiment, the planarization surface 51 comprises polyimide tape with a silicone adhesive backing applied to the PCB 1 prior to IC integration. This polyimide planarization surface 51, or any pressure sensitive adhesive, have the advantage of conforming to small imperfections in the surface 50 of the PCB 2 while still maintaining a good seal with the surface 6 of the IC 1. However, other approaches may be used. For example, the planarization surface 51 could be a thin elastomer sheet placed on a flat backing surface. In another embodiment, the backing surface could have pockets to accommodate protruding components on the circuit board.

This process permits many possibilities and variations. For example, more than one integrated circuit may be placed into the cutout 52, or multiple cutouts could be used to accommodate multiple ICs. Other components having a substantially planar face could be placed into the cavity, such as optical waveguides, light pipes, sensors, emitters, heaters, magnets, mirrors, optical filters, lenses, and the like. Electrical connections may be made via wire bonds, flip chip techniques (such as ball bonding, stud bonding, conductive polymers, or other suitable assembly techniques). In another embodiment, the PCB 2 has a pocket rather than an through-hole cutout, and the liquid polymer is injected through an opening in the flat plate. In another embodiment, the IC has connection pads on the bottom side, and these pads are wire-bonded, tape-bonded, or otherwise attached to the circuit board prior to the liquid polymer being injected. Once the planarization process is complete, wirebonds 3 can be applied and even encapsulated for robustness.

Many advantages are apparent from the foregoing description. The substantially planar surface of the resulting assembly permits leak-free interfacing to the fluidic unit 12. This attachment may be done with pressure-sensitive adhesive (PSA) 60. In one embodiment, the PSA 60 has fluidic channels patterned into it.

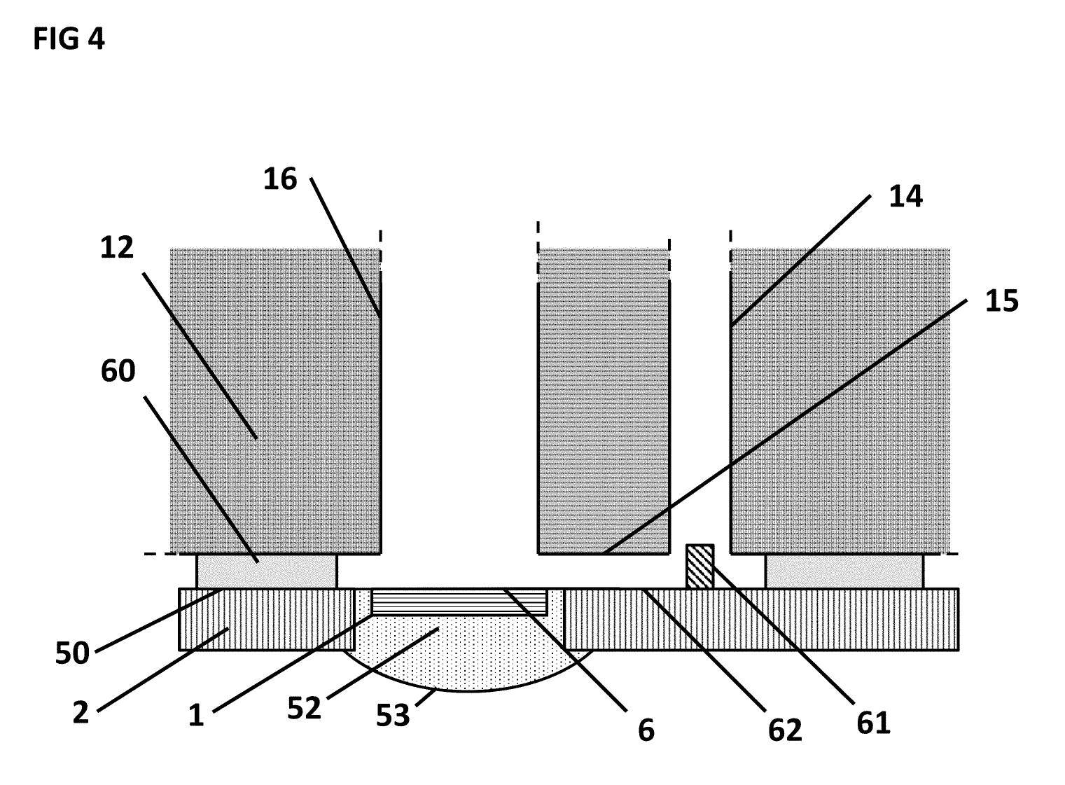

FIG. 4 is a cross sectional side view of the surface capillary 15 constructed from PSA 60. The surface capillary 15 can be cut, punched or milled into PSA 60 or transfer adhesive. The PSA 60 can provide a hermetic seal. The PSA 60 or transfer adhesive can be less than 250 μm, for example between 1 and 10 μm, or 10 and 25 μm, or 25 and 50 μm, or 50 and 100 μm or 100 and 250 μm. A thinner PSA 60 reduces the void volume of the surface capillary 15 and can bring the filtrate 13 in closer proximity to the surface 6 of the IC 1 for on-chip pre-treatment of the filtrate 13. A thicker PSA 60 can be used to ensure a hermetic seal despite non uniformity in the planarization surface.

Another advantage of the planarization process is compatibility with existing fabrication tools and methods; all of the required manufacturing unit operations are widely used for IC assembly. Another advantage of the planarization process is the possibility of integrating optical and other components into a multi-chip package, while readily permitting electrical, fluidic, and optical interconnections between the various components.

In order to use passive fluid flow, the surface 50 of the PCB 2 may be coated with a suitable coating to ensure the surface is hydrophilic. Numerous suitable coatings are commercially available. A protein adhesion layer may be coated the active surface 62 that is exposed to filtrate 13. The active surface 62 can be the surfaces under the sedimentation capillary 16, surface capillary 15 and delivery capillary 14. The active surface 62 can be the surface 6 of the IC 1, or the surface 50 of the PCB 2 or a combination thereof. The active surface 62 can also include a region over the resin 53 that has been planarized. Proteins or other reagents may be dried on the active surface 62 that is exposed to filtrate or other fluids.

In another embodiment, features on the PCB 2 may be intentionally raised above the active surface 62 to form a fluidic bridge 61 to ensure reliable capillary flow from the delivery capillary 15. An aspect of the present disclosure is a bridge 61 that ensures reliable flow of the filtrate 13 to the active surface 62 by sustained capillary action. Many common fabrication features can interfere with passive capillary action. A particularly problematic feature can be where a vertical capillary interfaces with a surface. Defects such as flash from injection molding can create a barrier feature that prevents liquid from exiting the vertical delivery capillary 14 due to the liquid's surface tension. Furthermore, the minimum diameter of the vertical capillary must be sufficiently large to allow the gravity-formed meniscus to cross any gap to the surface and promote flow. This constraint can increase the dead volume of the device, in turn increase the minimum sample volume and required filter capacity. The present disclosure addresses this difficulty by adding a bridge 61 to ensure reliable capillary action. The bridge 61 may be realized as several possible embodiments. In one embodiment, the delivery capillary 14 may be filled with packing material that touches the active surface 62. In this case, the packing material acts as the bridge 6. Such packing material may include glass or plastic particles, nitrocellulose, glass or polymer fibers, or other fibrous materials. The packing material can be impregnated with surfactants, salts, or other excipients to improve wicking characteristics. In order to form contact with the active surface 62 and promote, the bridge 61 can penetrate the bottom plane of the delivery capillary 14, and may contact the active surface 62. The fluid or filtrate in the delivery capillary 15 can be wicked to the active surface 62 by the bridge 61. Once the fluid or filtrate has wicked onto the active surface 62 by the bridge 61, the surface capillary 15 can sustain flow into the sedimentation capillary 16.

Another embodiment uses an insert as the bridge 61. The insert may be made of any suitable material, such as plastics, rubber, metal, glass, nitrocellulose, or any other suitable material. The insert may be placed loose into the delivery capillary 14, or it may have features to prevent it from freely moving in the delivery capillary 14 while still allowing liquid to fill the annulus between the insert and the capillary wall. Such features may include ribs, adhesives, press-fit interfaces, or other suitable methods.

Another embodiment uses one or more protrusions adjacent to the delivery capillary 14 to form the bridge 61. The protrusions extend the wall of the delivery capillary 14 to contact the active surface 62. These protrusions may be injection molded, attached by welding or adhesives, or by deforming the material forming the delivery capillary 14 assembly to form a lip. In an embodiment, the protrusions are made flexible to deform when the delivery capillary 14 is placed on the active surface 62. This automatically compensates for variations in PSA 60 thickness and ensures the bridge 61 touches the active surface 62.

Another embodiment uses a raised feature on the active surface 62. The raised feature should have sufficient height to nearly touch or penetrate the plane passing through the bottom edge of the delivery capillary 14. This variation avoids interference between the delivery capillary 14 and the active surface 62 even when the PSA 60 thickness varies significantly. The raised feature may be fabricated by depositing material (such as a UV-curable adhesive), press-fitting or gluing an insert into the active surface 62. If the active surface 62 is formed by the PCB 2, PCB features (such as a metal pad) may be used to form the raised feature. This feature may optionally be used to make an electrical connection to the fluid, for example for fill detection or electrochemical measurements by appropriate circuits connected to said pad.

Optical detection of magnetic particles 5 requires a source of incident light at the top of sedimentation capillary 16. To minimize assembly cost, it is desirable to locate the light source 70 on the printed circuit board 2 along with other surface mounted components. This light source may be a light-emitting diode. In one embodiment, this light source is a surface-mounted light-emitting diode. A light pipe 71 directs light from light emitting diode 70 to the top of the sedimentation capillary 16 by the principle of total internal reflection, where it is then projected down to the sensor 4.

Light pipe 71 may be fabricated of any suitable transparent material having a refraction index higher than that of air, such as clear acrylic. Optional inlet and outlet lenses may be integrally formed into the light pipe 71, improving the optical efficiency of the light pipe. Light pipe 71 may serve a secondary function of retaining the dry reagent spheres 17 in the fluidic unit 12. For that purpose, light pipe 71 may have a snap-fit, adhesive, or other suitable attachment to the fluidic unit 12. Light pipe 71 may optionally incorporate an optical splitter, such that light output from one light source 70 is incident upon two or more sedimentation capillaries 16.

Any elements described herein as singular can be pluralized (i.e., anything described as “one” can be more than one). Any species element of a genus element can have the characteristics or elements of any other species element of that genus. The above-described configurations, elements or complete assemblies and methods and their elements for carrying out the disclosure, and variations of aspects of the disclosure can be combined and modified with each other in any combination.

We claim:

1. An assay system comprising:
a printed circuit board;
an integrated circuit mounted on the printed circuit board, wherein the integrated circuit comprises a functionalized surface for specifically binding to magnetic particles, and wherein the integrated circuit comprises sensors for detecting the magnetic particles specifically bound to the functionalized surface;
an energy source configured to emit energy onto the functionalized surface, and wherein the sensors are configured to detect the energy;
wherein at least a part of the functionalized surface of the integrated surface is coplanar with a surface of the printed circuit board.

2. The system of claim 1, wherein an edge of the functionalized surface of the integrated circuit forms a seal with an edge of the surface of the printed circuit board.

3. The system of claim 1, further comprising a first separation conductor configured to produce one or more magnetic separation force to remove non-specifically bound magnetic particles from the sensors.

4. The system of claim 1, further comprising a second separation conductor configured to produce one or more magnetic separation force to remove non-specifically bound magnetic particles from the sensors.

5. The system of claim 1, wherein the printed circuit board comprises a fluidic bridge.

6. The system of claim 1, wherein the sensors comprise optical sensors.

7. The system of claim 1, wherein the energy source comprises a light source.

8. An assay system comprising:
a printed circuit board;
an integrated circuit comprising a functionalized surface for specifically binding to magnetic particles, and wherein the integrated circuit comprises sensors for detecting the magnetic particles specifically bound to the functionalized surface;
an energy source configured to emit energy onto the functionalized surface, and wherein the sensors are configured to detect the energy; and
a planarization surface on the functionalized surface and the surface of the printed circuit board.
